(19) United States
(12) Patent Application Publication
(10) Pub. No.: US 2007/0022905 A1
(43) Pub. Date: Feb. 1, 2007

(54) Method of Recycling Visco-Elastic Foam and Compressible Cushion Product Formed Thereby

(75) Inventor: Gary L. Allen, Wexford, PA (US)

Correspondence Address: BLYNN L. SHIDELER;THE BLK LAW GROUP, 3500 BROKKTREE ROAD, SUITE 200, WEXFORD, PA, 15090, US

(73) Assignee: MATERIAL INNOVATIONS LLC, Wexford, PA (US)

(21) Appl. No.: 11/458,121

(22) Filed: Jul. 18, 2006

Related U.S. Application Data
(60) Provisional application No. 60/703,154, filed on Jul. 28, 2005.

(51) Int. Cl. A61L 15/32

(52) U.S. Cl. 106/122

(57) ABSTRACT

A method of recycling memory foam includes shredding scrap memory foam into suitable particle sizes, mixing the shredded memory foam with high resilience and/or commodity flexible foam having comparable particle sizes, wherein the mixture is 20 to 80% by weight of shredded memory foam, and inserting the mixture into the liner of a cushion product, such as a pet bed. The liner and contained foam particle mixture may be inserted into an outer shipping cover and compressed for shipping and or storage. Further the shipping cover may be removed and the liner and foam particle mixture placed into an outer cushion cover prior to the liner returning to the full non-compressed state.

### RELATED APPLICATIONS

This application claims the benefit of pending U.S. Provisional Patent Application Ser. No. 60/703,154 entitled Method of Recycling Visco-Elastic Foam and Compressible Cushion Product Formed Thereby filed Jul. 28, 2005.

### BACKGROUND OF THE INVENTION

1. Field of the Invention

The present invention relates to a method of recycling visco-elastic foam and compressible cushion products formed thereby. More particularly the present invention is directed to a pet bed formed, in part, by recycled visco-elastic foam.

2. Background Information

Visco-elastic foam, also known as memory foam and visco-foam, is a heat sensitive, flexible polyurethane foam. At room temperature memory foam will mould itself to the shape of the body within a few seconds. The foam also holds its shape temporarily—a hand pressed into memory foam will leave a clear impression when removed. Memory foam was developed for NASA in the 1970's, although it really only hit the consumer market in mattress topper pads, and later in memory foam mattress, in the early 1990s. Ironically memory foam, as such, was never used in any space mission. The initial memory foam of the 1970's off-gassed too much for use in a closed environment. Memory foam has shown to be unique in the way it responded to temperature and also in ability to spread pressure over a greater surface area, and thereby reduce pressure points in patients significantly. The first company to really realize the potential of memory foam for the consumer market was Tempur-Pedic International, Inc. who created their own version of memory foam. Initially this was used in medical settings, but soon reports came back that patients who used the foam slept better, had less back pain or other pains in the morning, etc. Soon thereafter other consumer products using visco-elastic foam were introduced. As the years went by, memory foam became more and more popular, and now manufacturers all over the world are making their versions of memory foam. In addition to mattresses and toppers for mattresses, memory foam has been used in pillows and even pet bedding, essentially in the same manner as has been used in a regular mattress. These products have one or more layers of the memory foam generally with an underlying supporting layer or structure.

There are a couple of reasons that memory foam is so unique. One is the way memory foam cells deform when under a load. Unlike standard foams with high resiliency (i.e., that compress, but want to spring back to their original shape immediately), memory foam will compress and take a relatively long time to return to its original shape after the load has been removed. The polymer morphology of the visco-elastic is such that its soft-segment glass transition temperature is around room temperature. That lowers its resilience dramatically and makes it very slow to recover. The high resilience foam has its glass transition temperature at around −30 C, therefore, it would have visco-elastic properties only at that temperature.

Further visco-elastic foam is not suitable for the usual applications for scrap foam, such as rebond carpet underlay, because high resiliency is needed. Combined with the popularity of consumer products made from visco-elastic foam, there is an imbalance between the amount of scrap produced and practical uses for it. There is a need to develop effective and efficient techniques for recycling scrap memory foam.

In addressing the problem of recycling scrap memory foam, the present invention addresses the construction and shipping issues associated with cushion products, namely pet bedding. In the shipping and storage of cushion products there has historically been a problem that the shipping and storage costs are inappropriately increased as the manufacturer is essentially shipping a great deal of air. One solution to this problem has been proposed in published U.S. patent application 2003/0151295, which proposes a method that includes placing the cushion product, specifically a chair, in a vacuum chamber in order to suction out the air from inside the chair. The chair and accompanying vacuum chamber are then placed inside a storage container.

In addressing the problem of recycling scrap memory foam, the present invention also relates to pet bedding as suggested above. Pet bedding has been formed in a number of distinct ways, most commonly as layers of foam material, including incorporating memory foam layers as noted above. U.S. Pat. No. 6,196,157 discloses a pet bed made from layers of cut foam that is designed for stacking and easy transport. Other pet bedding materials have been proposed, including forming the pet bedding from recycled cellulose material (see U.S. Pat. No. 6,276,619) and cottonseed (see U.S. Pat. No. 6,698,380).

One object of the present invention is to provide an efficient and effective method of recycling visco-elastic foam. A further object of the invention is to provide a compressible cushion product formed, in part, by visco-elastic foam. A further object of the present invention is to provide a pet bed formed, in part, by recycled visco-elastic foam that decreases transportation and storage costs.

### SUMMARY OF THE INVENTION

It is noted that, as used in this specification and the appended claims, the singular forms “a,” “an,” and “the” include plural referents unless expressly and unequivocally limited to one referent. Further, the term “memory foam” in this application refers to visco elastic flexible polyurethane foam, such as sold under the Tempur® brand, but not limited thereto as discussed above. The term “scrap memory foam” refers to post-consumer memory foam, virgin scrap memory foam and mixtures of the two. The term “Conventional foam” within the meaning of this patent application refers to High Resilience Flexible Polyurethane Foam, Commodity Flexible Polyurethane Foam and mixtures thereof.

For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The various embodiments and examples of the present invention as presented herein are understood to be illustrative of the present invention and no restrictive thereof and are non-limiting with respect to the scope of the invention.

According to the present invention, a method of recycling memory foam is provided comprising:
a) shredding scrap memory foam into suitable particle sizes;
b) mixing the shredded memory foam with conventional foam having comparable particle sizes, wherein the mixture is 20 to 80% by weight of shredded memory foam; and
c) inserting the mixture into the liner of a cushion product.

In one embodiment of the present invention the particle size of the shredded memory foam is no greater than 1″ in any dimension. Particle size of the shredded memory foam of no greater than ¾″, ½″ and ¼″ in any dimension have also been proposed, respectively. There is a balance between the maximum size of the particles and the cost of manufacturing the shredded memory foam material.

In one embodiment of the present invention the mixture of the memory foam with the conventional foam in this application, is 20 to 80% by weight of shredded memory foam, as noted above. In one embodiment of the present invention the conventional foam is shredded and the shredding of the memory foam, the shredding of the conventional foam and the mixing of the shredded products may be performed simultaneously by feeding the product to the shredding equipment in the correct ratios. The conventional foam may be scrap conventional foam formed of post consumer products, virgin scrap conventional foam and mixtures thereof. The mixture of the memory foam with the conventional foam may be 40 to 60% by weight of shredded memory foam, and more specifically 50% by weight of shredded memory foam. This ratio will affect the overall weight of the cushion product due to the difference in densities of the foam particles, the overall compressibility of the cushion product and the operational characteristic of the resulting cushion product and can be adjusted as desired. However, the above described ranges provide the best resulting product characteristics.

In one embodiment of the present invention following the inserting of the foam particle mixture into the liner, the liner and contained mixture is inserted into an outer shipping cover, and the outer shipping cover, when sealed, is air-tight. The outer shipping cover, liner and foam particle mixture may be compressed by vacuum and/or mechanical compression with the compressed assembly packaged in a shipping container for shipment. The shipping cover, and possible the shipping package to some extent, are used to maintain the compressed position of the assembly during shipping and/or storage. Following shipment of the reduced volume package, the shipping cover can be removed and the liner and foam particle mixture placed into an outer cushion cover prior to the liner returning to the full non-compressed state. The visco-elastic material is not highly resilient and provides this advantageous delay in the liner returning to its shape.

In one embodiment of the present invention the cushion product being formed is a pet bed or pet bedding, which includes a removable, washable an outer cushion cover. The liner and foam particle mixture will be placed into the outer cushion cover.

These and other advantages of the present invention will be clarified in the description of the preferred embodiments taken together with the attached figures.

### BRIEF DESCRIPTION OF THE DRAWINGS

FIG. 1 is a schematic flow chart of the method of recycling scrap memory foam according to the present invention and the cushion product formed thereby;

FIG. 2 is a perspective view, partially in cross section of a cushion product, particularly a pet bed, made in accordance with the method of the present invention; and

FIG. 3 is a perspective view, partially in cross section of a cushion product, particularly a pet bed, without the outer cover and contained in a shipping cover and placed in a compressed shipping and storage state in accordance with the method of the present invention.

### DESCRIPTION OF THE PREFERRED EMBODIMENTS

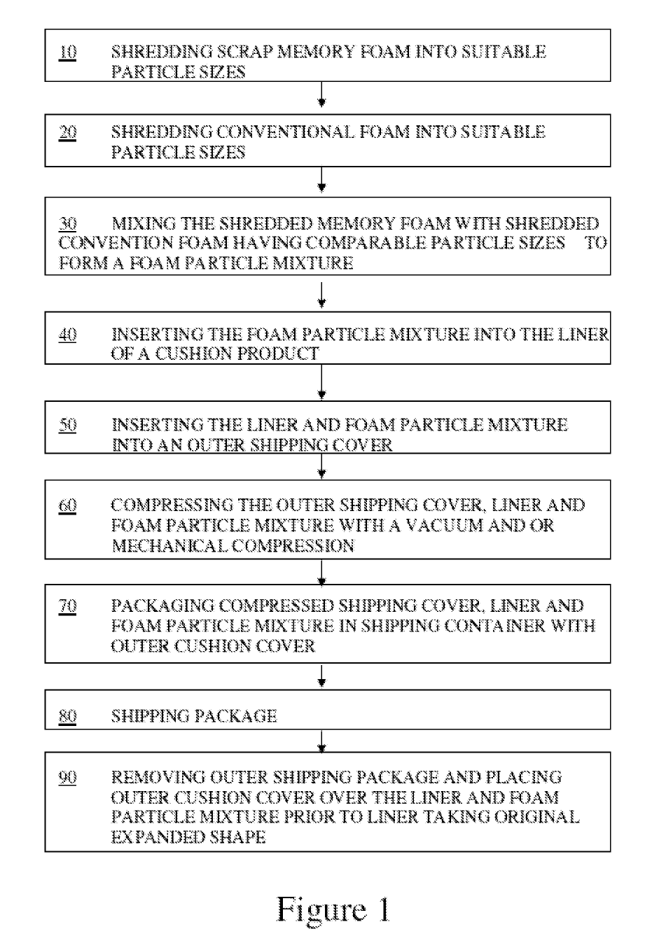

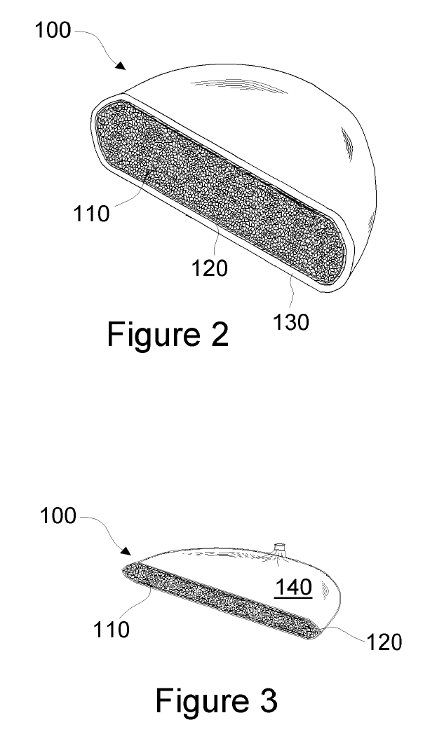

FIG. 1 is a schematic flow chart of the method of recycling scrap memory foam according to the present invention. The cushion product 100 formed thereby, such as a pet bed, is shown in FIGS. 2-3.

The initial step 10 of the recycling method according to the present invention is the shredding of the scrap memory foam into suitable particle sizes. Suitable particle sizes within the meaning of this application will refer to particles that have dimensions (length, width, height, or diameter) less than 2″. In one embodiment of the present invention the particle size of the shredded memory foam is no greater than 1″ in any dimension. Particle size of the shredded memory foam of no greater than ¾″, ½″ and ¼″ in any dimension have also been proposed, respectively in accordance with the present invention. There is a balance between the maximum size of the particles and the cost of manufacturing the shredded memory foam material. It is believed that particle sizes having maximum dimension between ¼″ and 1″ provide the optimum balance for forming efficient cushion products in the present invention.

The shredding of the memory foam in step 10 must take into consideration the properties of memory foam. For example, a suitable temperature reduction of the memory foam can allow almost any conventional shredding equipment to be used. However there is a higher cost associated with this additional temperature reduction step. Higher end shredding equipment may not require the temperature modification to the scrap memory foam and can eliminate the temperature modification step.

Step 20 of the recycling method according to the present invention is the shredding of the conventional foam, formed of high resilience foam as might be found in automobile seat cushions, or a lower priced commodity flexible foam as might be found in lower priced furniture, or mixtures thereof, into suitable particle sizes. The conventional foam may be a flexible polyurethane foam as known in the art. The key to the present invention is that the conventional foam is not visco-elastic. It is believed that if a high proportion of the conventional foam is high resilience foam (HR foam), the foam mixture probably performs better. Suitable particle sizes within the meaning of this application will refer to particles that have dimensions (length, width, height, or diameter) less than 2″. Preferably the particle sizes of the conventional foam will generally conform to those of the shredded memory foam. In one embodiment of the present invention the particle size of the conventional foam is no greater than 1″ in any dimension. Particle size of the conventional foam of no greater than ¾″, ½″ and ¼″ in any dimension have also been proposed, respectively in accordance with the present invention, similar to the memory foam particles discussed above.

The memory foam particles and the conventional foam particles are mixed to form a homogeneous foam particle mixture 110 in step 30. In one embodiment of the present invention the mixture 110 of the memory foam with the high resilient foam, also called conventional foam, is 20 to 80% by weight of shredded memory foam. More preferably, the mixture 110 of the memory foam with the high resilient foam may be 40 to 60% by weight of shredded memory foam, and more specifically 50% by weight of shredded memory foam. This ratio will affect the overall weight of the cushion product 100 due to the difference in densities of the foam particles. Further, the overall compressibility of the cushion product 100 is essentially determined by this ratio. The operational characteristic of the resulting cushion product 100 and can be adjusted as desired through modification of this ratio. However, the above described ranges provide the best resulting product characteristics, particularly for a pet bed type product 100.

The foam particle mixture 110 of shredded memory foam particles and conventional foam particles is inserted within a cushion liner 120 at step 40. The liner 120 is a conventional cushion product interior liner and is well known in the art. The details of the liner 120 construction need not be detailed further in this application. Further, other additives may be added to the mixture 110, as desired, such as cedar chips, anti-fungal material, anti-mold material, anti-microbial material, desiccant material, perfumes, aromatic oils, and the like. These additives are not in an amount that would alter the cushion characteristics of the mixture 110, or the range for the shredded memory foam particles.

In one embodiment of the present invention following the inserting of the foam particle mixture 110 into the liner 120 in step 40, the liner 120 and contained mixture 110 is inserted into an outer shipping cover 140 at step 50. The outer shipping cover 140 may be a vacuum packing bag, as known in the art, which is gas impermeable. The liner 120 is typically not gas impermeable as such would effect the characteristics of the cushion product 100, if it were then an access opening into the liner 120 would need to be used for the compression. The cover 140, liner 120 and foam particle mixture 110 may be compressed by vacuum and/or mechanical compression as shown in step 60. Vacuum compression is believed to be advantageous and consists of attaching a vacuum source to the shipping cover 140, also called a vacuum bag, and reducing the pressure within the cover 140 and liner 120, resulting in an associated compression of the mixture 110. At the desired level of compression the cover 140 is sealed to hold the assembly in the compressed condition, until the cover 140 is opened and/or removed. Mechanical compression is similar in that the cover 140, liner 120 and mixture is mechanically compressed (e.g. rollers) and the cover 140 sealed to hold the compressed state. The mechanical compression may be used to supplement the vacuum compression. The vacuum compression is believed to better accommodate the pressure differentials encountered in air transport (i.e. the holds of cargo planes are not fully pressurized and experience large air pressure differentials. Specifically, the cargo holds are typically pressurized to the 8000 or 10000 ft equivalent.

The compressed assembly is packaged in a shipping container (not shown) for shipment in step 70. The compressed assembly may be folded and secured with tie down straps (not shown) to assist in packaging in conventional shipping containers. The shipping cover 140 is used to maintain the compressed position of the assembly during shipping and/or storage as discussed above. The shipping package also, to some extent, may be used to maintain the compressed position of the assembly during shipping and/or storage. Following shipment of the reduced volume package in step 70, the shipping cover 140 can be removed and the liner 120 and foam particle mixture 110 placed into an outer cushion cover 130 prior to the liner 120 returning to the full non-compressed state. The visco-elastic material in the mixture 110 is not highly resilient and provides this advantageous delay in the liner 120 returning to its non-compressed shape.

The outer cushion cover 130 is also of conventional construction and will have a mechanism (e.g. a zipper) for allowing the cover 130 to be removed and washed and/or replaced. The cover 130 is designed to accommodate the intended use, such as being in contact with an animal for a pet bed. The liner 120 and the cover 130 can take any desired shape, such as circular as shown, rectangular, oval, elliptical, and the like. For a conventional pet bed size for the product 110 the shape may be a circular disc 38″-48″ in diameter and 6-10″ in depth. In one test of the present invention six dog bed liners 110 (having general dimensions of 39-40″ in diameter and 8-10″ high) were filled with a mixture 110 of shredded scrap flexible polyurethane foam (memory foam) and conventional foam. A vacuum was utilized to reduce the volume sufficiently to easily fit the beds 100 in a 12″×12″×24″ box (the compressed liner 110 and the associated covers 130).

Whereas particular embodiments of this invention have been described above for purposes of illustration, it will be evident to those skilled in the art that numerous variations of the details of the present invention may be made without departing from the invention as defined in the appended claims.

What is claimed is:

1. A method of recycling memory foam is provided comprising:
a) shredding scrap memory foam into suitable particle sizes;
b) mixing the shredded memory foam with conventional foam having comparable particle sizes, wherein the mixture is 20 to 80% by weight of shredded memory foam; and
c) inserting the mixture into the liner of a cushion product.

2. The method of recycling memory foam according to claim 1 wherein the shredded memory foam is no greater than 1″ in any dimension.

3. The method of recycling memory foam according to claim 1 wherein the shredded memory foam is no greater than ½″ in any dimension.

4. The method of recycling memory foam according to claim 1 wherein the shredded memory foam is no greater than ¼″ in any dimension.

5. The method of recycling memory foam according to claim 1 wherein the mixture of the memory foam with the conventional foam is 40 to 60% by weight of shredded memory foam.

6. The method of recycling memory foam according to claim 1 wherein the conventional foam is high resilience foam and wherein the mixture of the memory foam with the high resilience foam is 50% by weight of shredded memory foam.

7. The method of recycling memory foam according to claim 1 further including the step of inserting the liner and contained foam particle mixture into an outer shipping cover.

8. The method of recycling memory foam according to claim 7 further including the step of compressing the outer shipping cover, liner and foam particle mixture.

9. The method of recycling memory foam according to claim 8 wherein the step of compressing the outer shipping cover, liner and foam particle mixture includes a vacuum.

10. The method of recycling memory foam according to claim 8 wherein the outer shipping cover, liner and foam particle mixture is shipped in a compressed state.

11. The method of recycling memory foam according to claim 8 further comprising the steps of removing the shipping cover and placing the liner and foam particle mixture placed into an outer cushion cover prior to the liner returning to the full non-compressed state.

12. The cushion product formed by the process of claim 1.

13. The cushion product of claim 12 wherein the cushion product a pet bed, which includes a removable, washable an outer cushion cover surrounding the liner and foam particle mixture.

14. A cushion product comprising
a. A removable outer cover;
b. An inner liner contained within the outer cover; and
c. A mixture of shredded memory foam and conventional foam having comparable particle sizes, wherein the mixture is 20 to 80% by weight of shredded memory foam.

15. The cushion product according to claim 14 wherein the shredded memory foam is no greater than 1″ in any dimension.

16. The cushion product according to claim 14 wherein the shredded memory foam is no greater than ¼″ in any dimension.

17. The cushion product according to claim 14 wherein the conventional foam is high resilience foam and wherein the mixture of the memory foam with the high resilience foam is 40 to 60% by weight of shredded memory foam.

18. The cushion product according to claim 14 wherein the conventional foam is high resilience foam and wherein the mixture of the memory foam with the high resilience foam is 50% by weight of shredded memory foam.

19. The cushion product according to claim 14 wherein the cushion product is a pet bed.

20. A pet bed cushion product comprising:
a. A removable outer cover for the pet bed;
b. An inner liner contained within the outer pet bed cover; and
c. A homogeneous mixture of recycled shredded memory foam and high resilient foam having comparable particle sizes, wherein the mixture is 20 to 80% by weight of recycled shredded memory foam.
